(19) United States
(12) Patent Application Publication
(10) Pub. No.: US 2005/0100878 A1
(43) Pub. Date: May 12, 2005

(54) Method for the quantitative determination of one or more compounds

(76) Inventors: Bengt Bjellqvist, Uppsala (SE); Jean-Luc Maloisel, Uppsala (SE); Ronnie Palmgren, Uppsala (SE); Jonas Astrom, Uppsala (SE)

Correspondence Address: AMERSHAM BIOSCIENCES;PATENT DEPARTMENT, 800 CENTENNIAL AVENUE, PISCATAWAY, NJ, 08855, US

(21) Appl. No.: 10/398,439

(22) PCT Filed: Oct. 2, 2001
(86) PCT No.: PCT/EP01/11410

(30) Foreign Application Priority Data
Oct. 2, 2000 (SE) 0003566-7

(51) Int. Cl. C12Q 1/00

(52) U.S. Cl. 435/4

(57) ABSTRACT

The present invention relates to a method for the quantitative determination of the amount of one or more biomolecules, such as proteins or polypeptides, in one or more samples by utilising sample unique tagging reagents. More specifically, the method comprises steps of providing at least two samples; reacting biomolecules present in each sample with a sample unique mass tagging reagent to provide sample unique mass tagged forms thereof; combining tagged forms present in each sample to provide a single sample; coseparating, from the resulting sample, a mix of mass tagged forms of each of said biomolecules into different fractions; subjecting, for each fraction, the mix to mass spectrometry to obtain a mass spectrum; and determining from signals in each mass spectrum, the amount of the biomolecule corresponding to the spectrum in at least one of said samples relative to the amount of the same biomolecule in at least one of the remaining samples. In an advantageous embodiment, the separation step is a gel electrophoresis step. In some cases, it may be advantageous to also include a step of digesting biomolecules, such as protein(s).

### TECHNICAL FIELD

The present invention relates to a method for the quantitative determination of the amount of each of one or more compounds (comp 1, 2, 3 etc, compound(s)) in one or more samples (sample I, II, III etc, sample(s)). The method utilises a combination of sample unique mass tagging reagents (tagging reagent I, II, III etc, tagging reagents), separation and mass spectrometry of the compounds to be quantified.

The compounds preferably are biomolecules with peptide structure, a nucleic acid structure etc.

### BACKGROUND

The screening of global gene expression at the protein level for tissues and cells in specific situations (proteomics) has become a central instrument in biological investigations. In proteomics one of the most interesting aspects is to be able to address the relative changes in protein expression between two different cell states. These states can be dependent upon changes in the natural environment of the living cell or induced by the addition of a drug. The result will be a change in the profile of compounds expressed or synthesised by the cells, thus the occurrence of one or more proteins can be altered. It follows that in order to efficiently monitor changes one need to be able to make a quantitative determination of a large part of the protein spectrum, for instance.

Previously this has been accomplished by subjecting individual samples to two consecutive separation principles (two-dimensional separations), typically isoelectric focusing (first dimension) and normal gel electrophoresis (second dimension) followed by quantification of the protein content in individual spots. During the nineties one has started to use mass spectrometric techniques in order to identify proteins in spots, for instance by digesting the protein in a spot and subjecting the obtained peptide fragments to reverse phase liquid chromatography linked to a mass spectrometer (LC-MS). By comparing the global protein expression pattern so obtained for the biological origin of the sample with a control or reference sample, one has been able to monitor global changes in the patterns.

One problem encountered using stained gels in the proteomics workflow is the limited possibility to generate quantitative data for the protein expression changes. Silver, Coomassie, or even Fluorescence staining generally give rather crude estimates on the protein concentration, with fluorescence being the best one. These staining and software evaluation methods can not meet the quantitation demands of proteomics work, which probably needs to be able to detect relative protein expression changes down to 10%.

Quantification of individual proteins in one sample relative to the corresponding proteins in another sample by the use of differentially: isotope tagged affinity reagents has recently been suggested (Aebersold et al., WO 0011208; and Gygi et al., Nature Biotechnology 17 (1999) 994-999; and Mann M., Biotechnology 17 (1999) 1000-1001). In this technique, proteins in a first sample are reacted with a first affinity-tagging reagent and proteins in a second sample with a second affinity-tagging reagent. The first and the second reagents have the same chemical constitution but are differentially isotope tagged. Thus proteins in the first sample become differentially isotope tagged compared to proteins in the second sample. After tagging, the two samples are mixed and digested to obtain tagged and untagged peptide fragments of the tagged proteins. All the tagged fragments are then isolated as a common fraction by using the affinity tag as a handle for an immobilised affinity counterpart to the tag. Finally mass spectrometry is used for
(a) identifying the protein from which an individual tagged fragment derives and
(b) determining the amount of this protein in the first sample relative to the amount of the same protein in the second sample.

The mass spectrometry set up used comprises a single liquid chromatography step preceding the mass spectrometry (LC-MS). Typically the LC step is reverse phase liquid chromatography in order to separate different peptide fragment that are tagged. This technology includes one essential drawback since it is not easily adapted for multiple samples, which is desired for certain applications. Furthermore, the use of the affinity tag entails additional process steps for the binding thereof to a protein-binding group via a linker molecule. Thus, this technology is relatively complex in terms of the number of reagents and reactions required, and is therefore not ideal for use in an automated procedure.

WO 0011208 is incorporated in its entirety by reference.

Münchbach et al (Anal. Chem. 2000 (72) 40474057) have described a method for quantification in which each of two liquid samples containing proteins are subjected to 2-D electrophoresis followed by separate digestion and differential isotope tagging of the fragment mixture obtained for each individual spot. Corresponding spots for the two samples are then mixed and analysed by mass spectrometry in order to identify a protein or proteins in an individual spot and its (their) amount(s) in one sample relative to the amount of the same protein(s) in the other sample.

In spite of this recent works, improvements are still required for the above-mentioned methods, for instance with respect to the quantification of co- and/or post-translational modifications of proteins, and with respect to sensitivity, reproducibility, resolution, protocol simplification etc.

It has been suggested to use isotope tagged mercapto reacting alkylating reagents as an aid in protein identification by mass spectrometry in samples containing a protein mix (Goodlett et al., Anal. Chem. 72 (2000) 1112-1118; and Sechi et al., Anal. Chem. 70 (1998) 5150-5158). In these protocols a single sample is reacted with the reagent concerned before separation by electrophoresis. Identification is by mass spectrometry of the individual protein-containing fractions obtained. This field has recently been reviewed (Lahm & Langen, Electrophoresis 21 (2000) 2105-2114).

The quantification of the relative amounts of individual tagged proteins for two metabolically and differentially isotope tagged cell samples has recently been described (Oda et al., Proc. Natl. Acad. Sci. USA 96 (1999) 6591-6596).

### SUMMARY OF THE PRESENT INVENTION

A first objective is to provide improvements in regard to one or more of sensitivity, reproducibility, resolution, protocol simplification etc of the above-mentioned quantification methods.

A second objective is to provide improved quantitative measurements of the expression level and/or the co- and/or post-translation modifications of one or more individual proteins.

A third objective is an improved method for relating
(i) a difference in the expression level of one or more proteins and/or
(ii) a difference in the co- and/or post-translation modification of one or more proteins
   to one or more differences
(a) between samples obtained from a cell or an organism that has been subjected to a differential external stimulus,
(b) between samples obtained from cells and organisms having differentially mutated genes,
(c) between samples obtained from an healthy versus a diseased individual or an individual to be tested for a disease (i.e. in diagnosis),
(d) between samples obtained for one and the same individual at different occasions (i.e. for monitoring the development or curing of a disease in an individual),
(e) etc.

The individuals mentioned above comprise living organisms, in particular single cells and multi-cellular organisms, including animals, such as avians, mammals, amphibians, reptiles, fishes etc and include humans and beetles. The cells referred may originate from a vertebrate, such as a mammal, or an invertebrate (for instance cultured insect cells), or a microbe (e.g. cultured fungi, bacterial, yeast etc). Included are also plant cells and other kinds of living cells, e.g. cultured.

More specifically, the objects above can be obtained by the present invention, which relates to a method for the quantitative determination of the amount of one or more biomolecules in one or more samples by utilising sample unique tagging reagents, which method comprises steps (a)-(f)
(a) providing at least two samples;
(b) reacting biomolecules present in each sample with a sample unique mass tagging reagent to provide sample unique mass tagged forms thereof,
(c) combining tagged forms present in each sample to provide a single sample;
(d) coseparating, from the resulting sample, a mix of mass tagged forms of each of said biomolecules into different fractions by a separation protocol which is based on a principle selected from the group that consists of differences in hydrophobicity, differences in charge; differences in isoelectric point (pI); and differences in molecular size;
(e) subjecting, for each fraction, the mix to mass spectrometry to obtain a mass spectrum;
(f) determining from signals in each mass spectrum obtained in step (e), the amount of the biomolecule corresponding to the spectrum in at least one of said samples relative to the amount of the same biomolecule in at least one of the remaining samples. Methods based on differences in charge are understood herein to refer to methods wherein a change in pH is utilised for adsorption/desorption, and includes anion exchange and cation exchange based methods, which are well known to the skilled in this field. One example of a separation method wherein pI is utilised is chromatofocusing, which is also a well known method. More examples of separation methods will be mentioned below.

Thus, the present invention provides a method for the quantitative determination of a biomolecule in a sample, which method does not require use of any affinity label as part of the reagent, as disclosed in the prior art. Accordingly, the present method is easier to operate and also more adapted to automation than prior art methods.

In the present application, the term “biomolecules” is understood to include proteins and fragments thereof, such as peptides and polypeptides, nucleic acids, such as DNA or RNA, etc. Below, the term “compound” will sometimes be used to refer to the biomolecules. In one embodiment, the present biomolecule(s) are protein(s) or polypeptide(s) and the reagent is capable of binding to primary amines, cysteine residues or tyrosine residues thereof. In an alternative embodiment, the biomolecule(s) are nucleic acid and the reagent capable of binding to cytosine or adenine residues.

The present method can also include a digestion step inserted between any one of steps (a)-(e), with the proviso that if the digestion step is between steps (c) and (d), then at least two separate separation protocols are used in step (d). Such digestion can then be the fragmentation of a protein into polypeptides. In one embodiment, a digestion step is located before step (c). This embodiment will differ from the above mentioned technology described by Aebersold et al in that here two samples comprised of tagged peptides will be combined, while Aebersold et al have described a method wherein the digestion will take place in a combined protein sample.

A digestion step can optionally be located between steps (d)-(e), preferably with two or more separate separation protocols in step (d). In yet another embodiment, a digestion step is located between steps (a)-(d), such as between steps (a)-(b), (b)-(c) or (c)-(d), preferably with two or more separate separation protocols in step (d).

In one embodiment of the present method, the number (n) of biomolecule(s) to be quantified is two or more. In a specific embodiment, the number (m) of samples provided in step (a) is three or more. An essential advantage of the present invention resides in the fact, that it is easily adapted for accommodation of several different samples at the time. This is possible since the tagging reagent is simpler than the previously disclosed, cf. e.g. with the affinity tag-linker-binding protein disclosed by Aebersold et al. The present tagging reagent will also allow a less costly quantification than the previously suggested methods.

In one embodiment, the present biomolecule(s) are digested enzymatically. In an alternative embodiment, they are digested by a digestive chemical.

In yet another embodiment of the present method, two or more separation protocols are used in step (d), and each of at least two of them are different and capable of separating the mixture of tagged forms derived from step (c) into fractions, in which the predominating one of the biomolecule(s) to be quantified (in tagged forms) or a digestion product thereof (in tagged forms) differs between the fractions.

In one embodiment, the above mentioned sample unique mass tags differ with respect to composition of elements. In an alternative embodiment, said sample unique mass tags differ with respect to isotope composition for at least one element, preferably with the composition of elements being the same.

In an advantageous embodiment for determination of an absolute amount of a biomolecule in a sample, one sample is a reference or a control sample, such as an internal standard.

As discussed briefly above, in one embodiment, step (d) of the present method includes a separation protocol in which the mass transport of the biomolecule(s) to be quantified or of the digestion products thereof is by an applied electric field, such as in electrophoresis, preferably 2D-electrophoresis. In a specific embodiment, step (d) includes a separation protocol in which the mass transport of the biomolecule(s) to be quantified or of digestion products thereof is by a liquid flow, such as in chromatography.

### DETAILED DESCRIPTION OF THE INVENTION

The present inventors have now realised that one or more of the objectives mentioned above can be met for the method defined under the heading “Technical Field” above by including steps (a)-(f) in the order given below with a possible digestion step inserted between steps (a)-e). The digestion step, if present, results in one or more digestion products (digestion product(s)) for at least one of said compound(s). By varying the order of the steps it will be possible to optimise the balance between important features, for instance those discussed in the context of the first and second objectives. This in particular applies when a digestion step is present in a position between step (a)-(e). See under the heading “Digestion step” below. Steps (a)-(f) are:
(a) providing at least two of said sample(s), possibly containing said compound(s);
(b) reacting said compound(s) or digestion-products thereof in each sample with a sample unique mass tagging reagent to transform each compound or a digestion product to sample unique mass tagged forms;
(c) mixing tagged forms derived from each sample to form a mixed sample;
(d) coseparating from the mixed sample a mix of mass tagged forms of each of said compound(s) or of mass tagged forms of a digestion product thereof into different fractions;
(e) subjecting the mix of each fraction to mass spectrometry to obtain a mass spectrum for the mix;
(f) determining from signals in each mass spectrum obtained in step (e), the amount of the compound corresponding to the spectrum in at least one of said samples relative to the amount of the same compound in at least one of the remaining samples;
   with the proviso that if there is a digestion step between steps (c) and (d) then at least two separate separation protocols are used in step (d).

The individual steps will be further described below.

Step (a). The Samples and the Compounds to be Quantified.

This step comprises providing two or more samples (sample I, II, III etc). The total number of samples may for instance be up to 5 or up to 10. The samples may or may not contain compounds to be quantified.

The samples are in the preferred case of biological origin. They may be derived from biological fluids, such as cell lysates or cell homogenates, tissue homogenates, fermentation supernatants, body fluids etc. The most important body fluids are blood-derived such as whole blood, serum and plasma, and lachrymal fluid, semen, cerebrospinal fluid (CSF), saliva, urine etc and include also any other liquid sample containing bio-organic molecules selected among proteins, carbohydrates, lipids, hormones etc. The sample may also be a fraction of components present in an original sample, which for instance can be of biological origin as defined above. For typical fractionation procedures see under the heading “Fractionation steps” below.

One or more of the samples, for instance sample II, may be a control sample or a reference sample, which contains a control or a reference amount of at least one of the compound(s) to be quantified.

The number (n) of compounds to be quantified is ≧1, such as ≧2, ≧3, ≧4, ≧5, ≧6, ≧7, ≧8, ≧9, ≧10 or ≧20, ≧30, ≧40, ≧50, ≧100, ≧200, ≧300 etc. Typically the samples also contain other compounds having a structure in common with the compound(s) to be quantified, for instance of peptide structure.

The compounds to be quantified in the samples typically contain a general structure that is common to all the compounds to be quantified. The structure can be polymeric with a variation between the different compounds. The most prominent example of a useful common structure is the peptide structure, for instance the polypeptide structure (≧3 amino acids in sequence). An alternative polymer structure is the nucleic acid structure (≧3 nucleotides in sequence). The invention is believed to have its greatest impact on the quantification of compounds exhibiting peptide structure such as polypeptide structure, for instance as present in proteins.

All the compounds to be quantified have a reactive group that enables incorporation of a mass tag by the use of a mass tagging reagent. See further under the heading “Step (b). Mass tagging reagents and tagging”. Illustrative reactive groups of potential interest are:
(a) mercapto groups, such as in cysteine and homocysteine residues;
(b) disulphide groups, for instance with each directly bound carbon being sp³-hybridised, and reactive disulphides,
(c) amino groups, such as in amino acid residues containing an extra amino group (ω-amino acid residues), for instance lysine, and in N-terminal amino acid residues, and nucleotide residues containing an amino group;
(d) carboxy groups, such as in amino acid residues containing an extra carboxy group, and in C-terminal amino acid residues;
(e) ester, anhydride, and acid halide groups;
(f) thioether groups, such as in methionine residues;
(g) hydroxy groups, such as in amino acid residues containing hydroxy and other hydroxy-containing monomeric units of polymeric structures of bio-organic compounds,
(h) aldehyde and keto groups,
(i) phosphate groups,
(j) sulphate groups,
(k) epoxy groups,
(l) etc.

The term “compound to be quantified” in the context of the invention encompasses one single untagged molecular entity, or a mixture of different untagged molecular entities that will coseparate in the separation protocols applied after the tagging step. In the case of mixtures, it is contemplated that steps (e) and (f) above will encompass quantification of one or more of the compounds in the mixture.

Step (b). Mass Tagging Reagents and Tagging.

This step comprises reacting each of the samples provided in step (a) with a sample unique mass tagging reagent that introduces in a predetermined and reproducible manner the same tag on the compounds to be quantified in a sample. The tags are unique in the sense that their masses are different for different samples. Reagent I/tag I is used for sample I, reagent II/tag II is used for sample II, reagent III/tag III is used for sample III etc. The tagging reaction thus means that for each sample there are formed tagged forms of the/those compound(s) that is/are present and one wants to quantify. In other words comp 1-I, comp 1-I, comp 2-1 etc are formed in sample I; comp 1-II, comp 2-II etc are formed in sample II; etc.

In certain variants of the invention there may be used two or more mass tagging reagents per sample for introducing different tags on compounds having different kinds of reactive groups.

There may be a digestion step between steps (a)-(b). In this case the sample unique tag is introduced on digestion products of the compound(s) to be quantified. The tagged forms are tagged digestion products obtained for at least one digestion product for each of the compound(s) to be quantified.

The expression “in a predetermined and reproducible manner” means that there is formed a predetermined amount of a tagged form of each of the compound(s) to be quantified or a digestion product thereof in relation to the amount of the corresponding compound in the sample provided in step (a). In preferred variants the mass tagging reagents and the conditions should be selected to give an essentially quantitative yield based on the corresponding untagged compound or untagged digestion product, where appropriate. Quantitative yield is e.g. all yields ≧80%, such as ≧90% or ≧95%.

In the preferred case the tagging reagents have been selected so that each tagging reaction can be run under essentially equal conditions and/or can give essentially the same tagging efficiency.

The differences in mass between the tags depend on (a) different elemental composition of the tags and/or (b) different isotope composition of one or more elements of the tags.

For tags complying with alternative (a), the difference shall preferably be such that the difference inseparation behaviour during the separation step (step (d)) is insignificant for the tagged forms of a compound or of a digestion thereof containing the tag. Groups such as hydrocarbon groups possibly containing amino, ether, thioether and hydroxy are believed to be potentially useful as tags according to this alternative.

Tagged forms of a compound or of a digestion product in which the tags are according to alternative (b) will inherently behave essentially equal in the separation protocols.

The tags should be small compared to the molecular weight of the compounds to be quantified. Most tags typically have a mass that is at most 50%, many times at most 10% such as at most 5%, of the mass of the heaviest of the compound(s) to be tagged (i.e. is either the corresponding untagged compound or the corresponding digestion product). Mostly the optimal mass tags have molecular weights ≦1000 daltons, such as ≦500 Dalton.

In both alternative (a) and (b) above, the difference in mass of the tags introduced should be such that it results in distinct measurable peaks in the mass spectra obtained in step (e). Calculations in step (f) will be facilitated and made more safe and reliable if the tags are selected such that base line separation of the peaks corresponding to the tagged forms of a compound or of a digestion product thereof is enabled. The optimal mass difference between the tags depends on various factors, for instance the mass spectrometer. The mass difference of two different tags of the same elemental composition is typically ≧4, such as ≧6 Dalton, for instance 7, 8, 9, 10, 11, 12 Dalton or ≧13 Dalton. An upper limit for this mass difference can be 25 Dalton. In case the tags differ with respect to elemental composition the difference may be up to 200 Dalton or higher. If three or more tags are used the largest mass difference should be within the limits given above.

Typical isotopes that are useful in the present invention are: ¹H/²H/, ¹²C/¹³C, ¹⁴N/¹⁵N, ¹⁶O/¹⁷O/¹⁸O, ³²S/³⁴S, isotopes of Cl, Br and I and P etc.

The mass tagging reaction preferably takes place in one step/reaction, but may also involve a step-wise reaction protocol including for instance a first activation step/reaction and a second step/reaction during which the groups causing the difference in mass are introduced. Depending on the circumstances, also further consecutive steps, reactions and reagents may be needed. All the steps and reagents involved in tagging a compound or a digestion product are comprised within the terms “mass tagging reaction” and “tagging reaction”. Similarly, the expression “mass tagging reagent” and “tagging reagent” include all the reagents needed for tagging a compound or a digestion product (except for the compound or the digestion product as such). In case the original compound or the digestion product does not contain any suitable reactive group permitting mass tagging such groups can be introduced in the mass tagging reaction.

Potential mass tagging reagents should have reactive groups matching reactive groups in the compounds to be quantified as outlined in the table below.

REACTIVE GROUP IN MASS TAGGING REAGENTS.

COMPOUND/
DIGESTION
PRODUCT

| | |
|---|---|
| Mercapto | Activated alkenes, such as α-β-unsaturated carbonyl groups, |
| | and α-halo carbonyl groups, such as α-iodo carbonyl groups, |
| | and disulphide groups, such as reactive disulphides. The |
| | carbonyl group or disulphide group binds further to a group |
| | that differs in mass for different reagents. Examples of α-β- |
| | unsaturated carbonyl reagents are differentially deuterated N- |
| | alkyl acryl or methacryl amides, various undeutetrated forms |
| | of N-alkyl acryl or methacryl amides in which the mass of |
| | the alkyl group differs etc. |
| Amino | Activated acids, for instance activated esters, acid halides, |
| | acid anhydrides etc. The acid moieties of the activated acid |
| | bind further to a group which differs in mass for different |
| | reagents, for instance differentially deuterated alkyl groups or |
| | alkyl groups of different elemental composition. |
| Carboxy | A carboxy group in a compound to be quantified or in a |
| | digestion product is preferably transformed to activated |
| | carboxy and combined with a mass tagging reagent |
| | containing an amino or a hydroxy group, for instance. The |
| | amino or hydroxy group in a tagging reagent may bind to an |
| | alkyl group that for different reagents have different isotope |
| | compositions and/or different elemental composition. |
| Thioether | Oxidation agents for transforming the thioether function to a |
| | sulfoxide or a sulphone. The mass tagging reagents are often peroxides |
| | containing different isotopes of oxygen. |
| Aldehydes, | Primary and secondary amines containing alkyl groups in |
| ketones | which the alkyl part differ in elemental and/or isotope |
| | composition. The adduct formed between the amine and the |
| | carbonyl group is reduced with for instance a borohydride. |
| Phosphate | Metal chelates in which the ligands differ with respect to |
| | elemental and/or isotope composition. |
| Hydroxy- | Halogen |
| phenyl | |

Thus, in the advantageous embodiment where the biomolecule which is quantified is a polypeptide or a protein, the reagent may bind to the N-terminal peptide or/and lysine therein. Examples of tag pairs of one deuterated and one non-deuterated can then be as below:
Labelling of the N-Therminal at Peptide or/and Lysine in the Peptide/Protein: 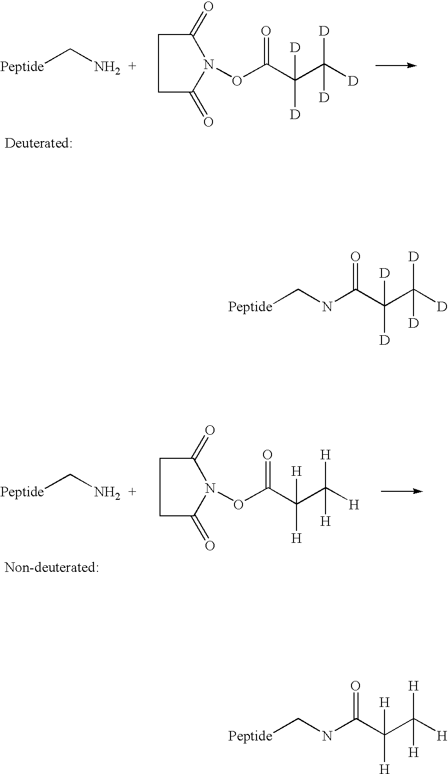
Labelling of Cysteine in the Peptide/Protein: 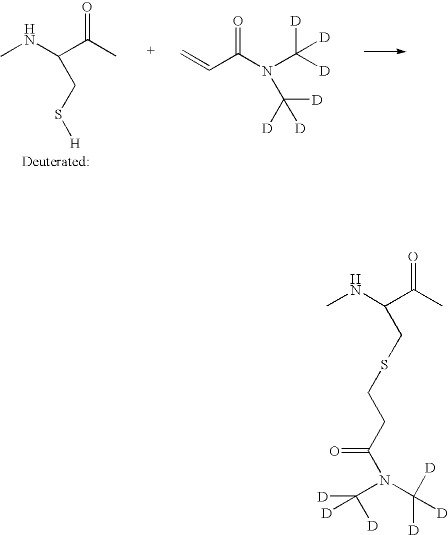 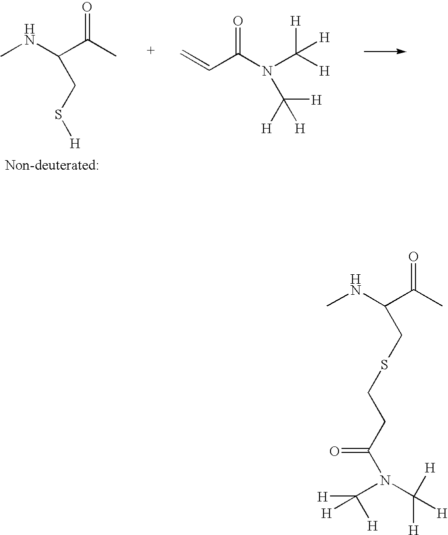
Labelling of the Tyrosine in the Peptide/Protein: 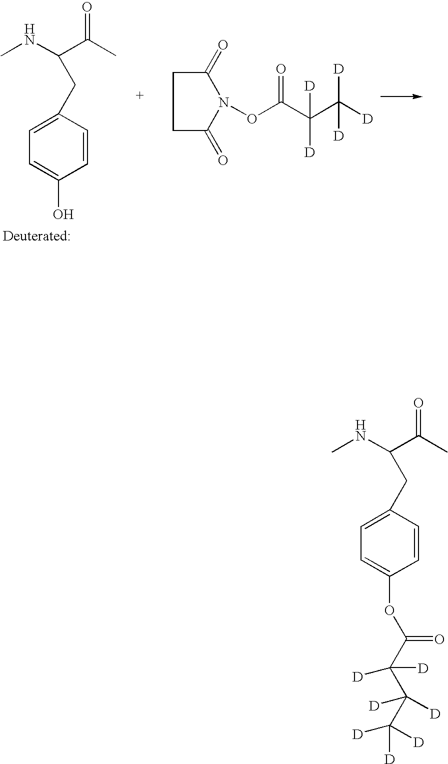 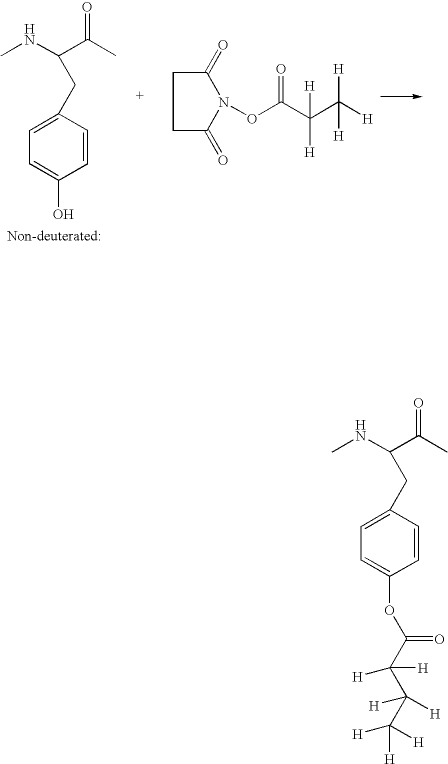
Labelling of DNA/RNA

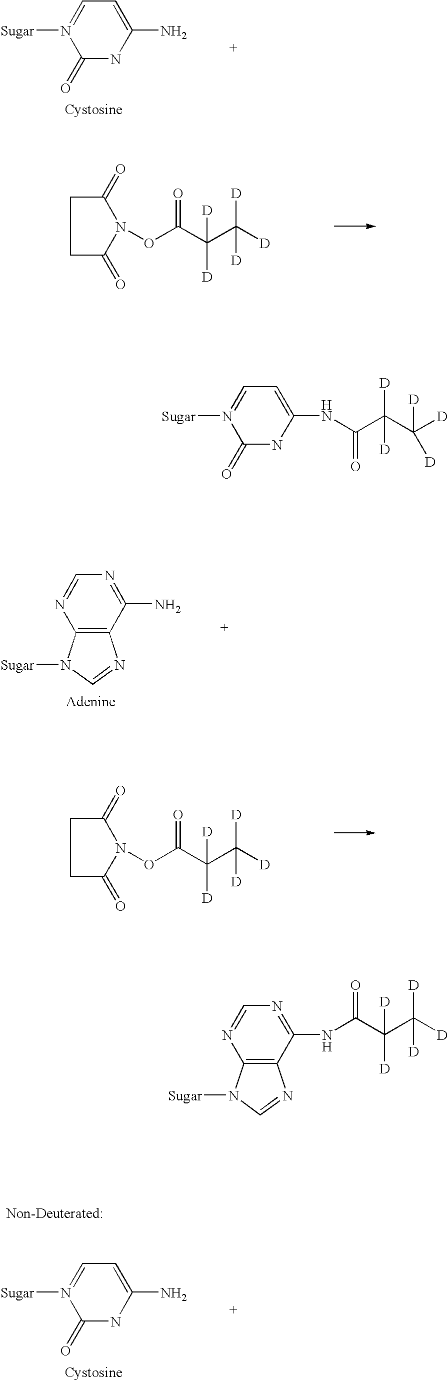

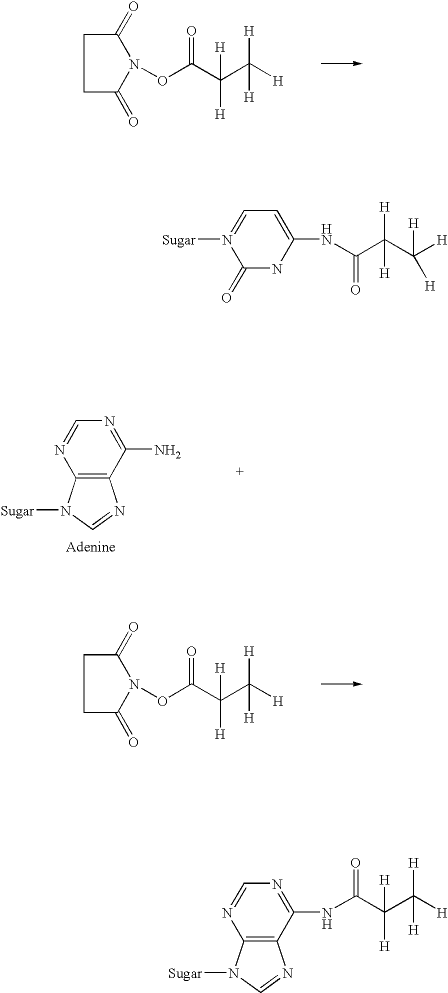

One especially advantageous use of the present method is for multiplex analysis, i.e. where multiple samples have been tagged with isotopic tagging reagents, which constitute a series of different content of the isotope. For example, the heavy isotope of an appropriate reagent may have a content of 2, 4, 6 etc deuterium atoms, while the light isotope thereof correspondingly will present the same variation of the number of H atoms. This allows comparing a large variety of samples at the same time. Since the labelling reagent is simpler it is easy and less expensive to diversify it.

For example, with the use of acrylamide derivatives of iminodiacetic acid, 7 different samples can easily be accommodated at a time (0, +3, +6, +10, +12, +16, +20).
Synthesis of Tri-Functionalised Scaffolds for Labelled Chemistry

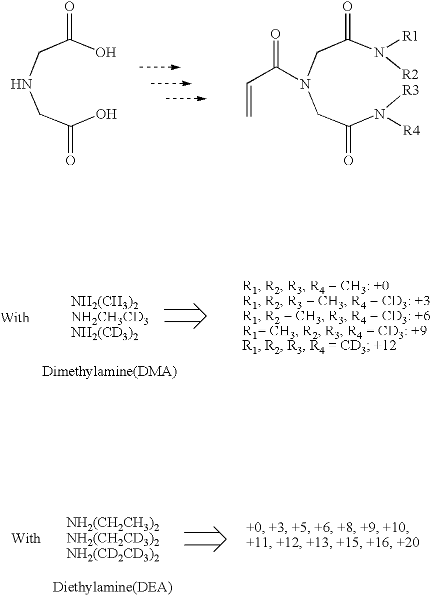

Still further combinations can be deduced from the chemical literature. See also Aebersold et al (WO 0011208).

Catalytic reactions, for instance enzymatic reactions and enzymes for introducing the mass tag at a predetermined position in the compounds to be quantified, are included in'the concept of mass tagging reactions/reagents. Predetermined positions in proteins can be a free internal or a free terminal carboxy group, a free internal or a free terminal amino group, a free internal hydroxy group etc.

Specific introduction of the tag to a predetermined position or group will permit quantification only if the compound to be quantified or a digestion product thereof has the reactive group that is capable of reacting with the mass tagging reagents used. In order to extend the quantification to other compounds that neither as such or as a digestion product has this group, a combination of different mass tagging reactions/reagents and/or different conditions will have to be applied to the same sample. It is believed that the simplest way to accomplish this is to perform the sequence (a)-(f) for each predetermined position or group on separate aliquots of the samples.

The mass tagging reaction should be run under conditions permitting reproducibility and predetermined amounts of tagged products. This typically means that there should be an excess of tagging reagent in relation to the sum of the total amount of compounds to be quantified or of digestion products thereof to be tagged plus the amount of other constituents in the reaction mixture that consume mass tagging reagents. Typically the excess should be at least 50%.

When there are mercapto groups in the compounds to be quantified, one preferably adds an excess of a reducing agent, to prevent disulphide formation between the compounds to be quantified. The reducing agent may be a low molecular weight thiol compound, typically dithiotreitol (DTI), mercapto ethylamine, mercapto ethanol etc, or a non-mercapto-containing reducing agent such as a phosphine like tributyl phosphine. The mass tagging reagent used then should be in excess of all mercapto groups present in the reaction mixture. In the case the compounds to be quantified are proteins they often contain disulphide groups in forms of cystine residues. The addition of the reducing agent then has the further effect of oxidising these groups to mercapto groups that can be used in the mass tagging reaction.

After the tagging reaction, it can be advantageous to remove the excess of tagging reagents. Depending on the circumstances this can be done on the individual reaction mixtures between steps (b)-(c), on the mixed sample between steps (c)-(d), or on the individual fractions after the separation, i.e. between steps (d)-(e). Removal can be accomplished by contacting the solution containing the excess with a solid phase bound form of structures that are capable of interacting with unreacted tagging reagents. The solid phase may for instance be in the form of beads and other particles.

The tags should not contain structural elements that give the same or similar fragmentation pattern in MS as the compound to be quantified. This in particular applies if tandem MS is used in step (e). For instance, if the compound exhibit peptide structure, such as polypeptide structure, then the tag should not exhibit this kind of structure.

Step (c). Mixing Samples.

This step comprises mixing defined aliquots of the samples treated in step ( ) to form a mixed sample. Preferably the aliquots are equal for each sample.

Step (d). Separation.

This step comprises coseparating the mass tagged forms of each compound or of a digestion product thereof into separate fractions by the use of one or more separate separation protocols.

After the separation step an essential part of each of comp 1-I, comp 1-II etc will be in fraction 1, an essential part of each of comp 2-I, comp 2-II etc in fraction 2 etc. The separation protocol(s), mass tags/tagging reagents (I, II etc) etc are thus adapted to each other and to the compound(s) (or digestion product(s) where appropriate), such that each fraction will contain a higher amount of a mix of tagged forms of one compound or of tagged forms of a digestion product of the compound and a lower amount of tagged forms of any of the other compounds or of any of their tagged digestion products. In the preferred case this means that the higher amount is more than three times the amount of tagged forms of any of the other compounds or of any of their tagged digestion products. Most preferably the lower amounts is insignificant or zero.

If there is one sole compound to be quantified, it suffices that the separation step leads to a fraction that has an increased content of tagged forms of this compound or a digestion product thereof relative to the other constituents of the mixed sample.

In certain preferred variants of the invention there are two or more separation protocols. In order to obtain a sufficiently high resolution it is advantageous that at least two of these separation protocols differ with respect to the separation principles employed.

Typical separation principles are separations based on differential interactions between the compounds and a surrounding medium. The differences may, for instance, be reflected by differences in size and/or charges of the compounds, such as in size exclusion chromatography and in adsorption chromatography, in gel electrophoresis e.g. PAGE, in capillary electrophoresis, in isoelectric focusing e.g. in gels, capillaries, etc. In specific embodiments of the present invention, the differences may also be reflected in a differential affinity of the compounds with one or more ligands attached to the separation media used etc. Differential affinity may be utilised in either or both of the adsorption step and the desorption step.

The concept of different principles is also defined by the way in which the mass transport is taking place during the separation. Illustrative examples are by stirring or by other means giving turbulence/agitation such as in batch-designed procedures, by liquid flow such as in chromatographic procedures, by an applied electric field such as in electrophoresis, by centripetal force such as in centrifugation, by gravity etc.

Separation protocols that are based on the same separation principles include that a separation protocol is run on a fraction obtained in a previous protocol under essentially the same conditions as in the previous protocol. It also includes variations between different protocols, such as changes in pH, concentration of salts and the like etc.

For adsorption methods the changes referred to above may be present in the adsorption and/or the desorption step.

The appropriate selection of separation protocols and combination thereof will depend on the kind and size of compounds to be quantified and when appropriate also the size of digestion product(s) if a digestion step is preceding the separation step (d). As a general rule a more complex mixture will require a total higher resolving capacity on the combination of protocols selected. Complex mixtures, for instance containing more than 20-100 compounds with a common structure including digestion products and being relatively small (≧4000 Dalton) and/or in a narrow size range, implies that separation protocols in principle solely relying upon size exclusion will be of minor interest. For this kind of situation it will be of preference to include at least one protocol based on for instance reverse phase or other ligands binding via some kind of affinity.

For larger molecules, protocols based on size exclusion can be used (gel electrophoresis, size exclusion chromatography etc), with preference in combination with high resolving techniques/protocols for instance based on differences in isoelectric points (where appropriate).

The separation step (d) may also include one or more of the various fractionation steps mentioned under the heading “Fractionation steps” below. However, it is imperative that a fractionation step at this position has to be combined with at least one, preferably two or more separation protocols, as discussed above.

Steps (e)-(f). Mass Spectrometry and Quantification.

These steps comprise subjecting each fraction to mass spectrometry. For each fraction this will give a mass spectra for the mix of mass tagged forms of one compound to be quantified or of a digestion product thereof. From the relation of the signal (peak) for the mass tagged forms which are present in the mix/fraction, contain the same number of tags and derive from the same compound, the amount of each form relative to any other of the forms can be determined. The relative amounts for the mass tagged forms in a mix can then give the relative amounts of corresponding compound in the original samples. The methods for doing this kind of calculations have been previously practised in the field. Compare Aebersold etal (WO 0011208); Gygi et al (Nature Biotechnology 17 (1999) 994999); Münchbach et al (Anal. Chem. 2000 (72) 40474057) and Oda et al (Proc. Natl. Acad. Sci. USA 96 (1999) 6591-6596).

The term “mass spectrum” in this context refers to the set of signals obtained in the mass spectrometer from the various tagged forms in a mix and also the representation of the signals as peaks in a conventional mass spectrum.

Typically the mass tagging reaction will introduce one, two three etc identical tags on each compound or on a digestion product thereof. For tagged forms containing one, two, three etc identical tags, the peaks to be used in a mass spectrum for the quantification in the invention will be positioned at mass differences that are the same as, twice, three times etc, respectively, the mass differences of the tag used.

In the case the salt concentrations in the fractions obtained in step (d) are too high for the mass spectrometry step, there may be included one or more desalting steps between the separation step (d) and the mass spectrometry step (e). As an alternative one or more of the individual preceding steps may be precautionary adapted to conditions giving a sufficiently low salt concentration in the individual fractions to allow for a high qualitative mass spectrometry step (f).

Depending on the kind of separation methodology immediately preceding step (e), LC-MS, CE-MS etc may be used in step (e). Tandem MS (MSⁿ) may be used in case there is a need to sequence the tagged peptide fragments that are detected in the mass spectrometer, for instance for identifying the compound corresponding to a tagged fragment detected in the mass spectrometer. This applies, for instance, if there is a fragmentation step before the separation steps removing all the untagged fragments. In the case the fractions obtained in step (d) contains also the fragments (digestion products(s)) obtained in a preceding digestion step, then the identification of the individual compounds can be assisted by the use the mass peaks (m/z) for the digestion product(s), possibly tagged, of each compound.

For ionisation the mass-spectrometry may utilise electrospray (ESI), matrix associated laser induced dissociation (MALDI) etc of the tagged forms in the individual fractions. Depending on the separation principle preceding the mass spectrometry step set up may be in form of LC-MS, CE-MS etc.

Commercially available mass spectrometers have computer programs that utilise known digestion peptide fragment pattern from chemical and/or enzymatic digestion of proteins and fragmentation patterns in mass spectrometers for identifying individual proteins. In principle this applies also to compounds of non-peptide structure. In the case a fraction obtained in step (d) contains mass tagged forms of a mixture of compounds, available mass spectrometers are capable of determining the mass spectrum for the mixes of tagged forms for each individual compound present in the fraction and also of identifying the compound(s). Mixtures of compounds that coseparate in step (d) have been discussed in the context of step (a).

The basis for the quantification is
(a) that the signal in a mass spectrum is a function of the amount of the tagged form applied in the mass spectrometer and
(b) that this amount in turn is a function of the amount of the untagged compound in the sample corresponding to the tag concerned.

When calculating the amounts in step (f) one has to take into account, for instance,
(1) the relative volumes of the samples provided in step (a) and/or the relative total concentration of the compounds to be quantified in the same samples,
(2) relative dilution between the different reaction mixtures in step (b),
(3) relative tagging efficiency between the various tagging reagents in step (b),
(4) relative volumes of samples mixed in step (c).

In a preferred case at least one of the following features applies:
(i) all the samples in step (a) have essentially equal volumes and/or the same total concentration of compounds to be quantified,
(ii) the dilutions and the tagging efficiency in the different reaction mixtures in step are essentially equal, and
(iii) the volumes of the reaction mixtures mixed in step (c) are essentially equal.

In case the compounds to be quantified have polypeptide structure, item (1) above means that it is preferred to take into account the relation between the total protein concentration of each of the samples provided in step (a). In the preferred variant, item (i) includes that the total concentration of protein in each of the samples provided in step (a) should be the same.

The Digestion Step

There may or may not be present a digesting step in which the compounds to be quantified are cleaved to fragments, for instance chemically or enzymatically.

If there is no digestion step, quantification will be based on the intact protein.

For compounds of biological origin, for instance exhibiting polypeptide structure and other biopolymers, chemical and enzymatic digestions are the two main alternatives:
(a) Chemical digestion which for instance can be used if the compounds exhibit polypeptide structure. A typical digestion reagent of this kind is CNBr that cleaves polypeptide structures at methionine residues.
(b) Enzymatic that typically can be used for bioploymers of the types mentioned above.

Typically examples of enzymes are trypsin, ArgC, AspN, GluC, LysC, V8 protease (D,E, V8 protease (E) etc. Most of the useful enzymes for proteins are endopeptidases. When selecting a cleaving/digestion agent it is important to select an agent that does not remove the tag and is capable of cleaving tagged forms of the compounds. Trypsin, for instance, can be used if the compound(s) to be quantified are proteins that contain arginine and/or lysine residues together with cystine or cysteine residues. If an internal free amino group of a protein is used for tagging, the enzyme used should not utilise an amino acid residue providing a free amino group for recognising its cleavage site.

For proteins, the digestion should result in peptide fragments having lengths in the range of from 8 amino acid residues and upwards. The exact number will depend on the individual protein and the digestion method used. Typically the upper limit is for instance in the interval 20-30 amino acid residues. The corresponding interval with respect to monomeric units may apply also to other of the above-mentioned biopolymers.

Two or more cleavage agents may potentially be used in combination, either simultaneously or consecutively.

Various proteases and their substrate specificity and the fragment pattern they will give for different known proteins are available in database form (e.g. ProFound and Swissprot).

If the digestion step is located between steps (a)-(b), the advantages will be that the digestion will open up the structure of complex compounds, for instance the bio-polymers discussed above. This is likely to lead to a better utilisation of the mass tagging reagents used in step (b). Compare for instance Munchbach et al (Anal. Chem. 2000 (72) 40474057) who utilised conditions allowing selective tagging of free amino terminals.

A positioning of the digestion step in front of the separation step (d) will lead to more complex mixtures to separate compared to placing the separation step between steps (d)-(e). This means that in order to obtain an improved resolution these positions of the digestion step preferably require two or more separation protocols in the separation step. High-resolution techniques, such as 2-D gel electrophoresis and other techniques utilising differences in molecular sizes, will be of less or none value. The mass spectra obtained will lack information of the digestion fragments separated into other fractions. The chances of discriminating between homologous proteins and co- and/or post-translation modifications will be reduced or made impossible.

There are advantages in having the digestion step between steps (b)-(c) compared to between steps (c)-(d) because the former variant will facilitate equal efficiency in the digestion of a compound independent on from which sample it derives. This in particular applies if there are large concentration differences of digestible compounds between samples.

If the digestion step is located between steps (c)-(d), the literature suggests insertion of a subsequent extra step (fractionation based on a preceding derivatisation permitting the fractionation; Aebersold et al., WO 0011208; and Gygi et al., Nature Biotechnology 17 (1999) 994-999).

If the digestion step is located between steps (d)-(e), the mixture to be separated will be relatively simple facilitating improved resolution in the separation step. The mass spectra will contain information of all parts of the compound to be quantified. This will facilitate resolution of homologous compounds, for instance the above-mentioned biopolymers, in the separation step, and study of co- and/or post-translation modifications from the mass spectra obtained. Identification will be possible with simpler mass spectrometer systems.

Fractionation Steps.

In the inventive method there may also be included so-called fractionation steps in which a starting sample is fractionated into subfractions containing cell constituents or groups of compounds. Typical subfractions are subcellular fractions, protein fractions, antibodies, certain enzymes etc.

This kind of fractionation steps typically involves at least one of precipitation, centrifugation, chromatography, adsorption, electrophoresis, partition between aqueous liquid phases etc. The steps may include well-known affinity principles such as between a ligand and a receptor, for instance based on electrostatic attraction between oppositely charged groups, hydrogen bonding, charge transfer, pi-pi-interaction, hydrophobic interaction etc. The affinity principles are divided into ion exchange and pure affinity including also bio-affinity and other affinities involving complex interactions such as between antibody and antigen/hapten, lectin and carbohydrate, immunoglobulin-binding proteins and immunoglobulins etc.

Application of fractionation steps in the field of the invention has previously been suggested and/or utilised (Aebersold et. al., WO 0011208; and Gygi et al., Nature Biotechnology 17 (1999) 994-99).

Other Derivatisation Steps

At certain positions in the sequence (a)-(f) there may also be inserted derivatisation steps for the purpose of
(1) enhancing the ionisation during the mass spectrometry step (e) (at any position preceding step (e)),
(2) enhancing the fragmentation during the mass spectrometry step (e) (at any position preceding step (e)),
(3) minimising separation differences introduced by the mass tag (at any position preceding step (d)), and
(4) introducing an affinity handle to be used in a fractionation step, for instance as suggested, etc

Other kinds of derivatisations are predictable.

Enhancement of ionisation can be accomplished if an acidic or basic group is inserted, e.g. —COOH, —SO₃H, primary, secondary or tertiary amino groups, nitrogen heterocycles, ethers or combinations of these groups. Also permanently charged groups can give this effect, e.g. quaternary ammonium groups, phosphonium groups, chelated metal ions, sulphonium groups, etc.

Enhancement of fragmentation can be accomplished by introduction of positively or negatively charged groups on for instance a terminal amino group. See for instance Keough et al (WO 0043972).

Introduction of an affinity handle to be used in a fractionation step can be carried out as outlined by Aebersold et al (WO 0011208) and Gygi et al (Nature Biotechnology 17 (1999) 994-999).

For each of these additional derivatisations the same reagent is preferably used for all the samples. A potentially important variant is to design the mass tagging reagent so that one or more of these derivatisations are accomplished when the mass tagging reaction is carried out. See for instance Aebersold et. al., WO 0011208; and Gygi et al., Nature Biotechnology 17 (1999) 994-999 in which an affinity handle and the mass tag is introduced simultaneously by the same reagent.

### Experimental Part

Synthesis of Isotope Tagged Acrylamide

N,N-d₆-Dimethylacrylamide (d₆-DMA)

N,N-d₆-dimethylamine (0.5 g, 5.71 mmol) and triethylamine (1.16 g, 11.4 mmol) were dissolved in 25 ml of dichloromethane during stirring. The flask was put in an ice-bath and acryloylchloride (0.516 g, 5.71 mmol) was added drop wise. A white salt was instantly formed. The salt was precipitated in −20° and thereafter filtered off and washed with cold dichloromethane. The solvent was evaporated and the crude product was purified by flash chromatography on silica gel (60 g, particle size 0.040-0.063 mm) with dichloromethane:acetone 85:15 as eluent. The product (transparent liquid) was analyzed by TLC and NMR. Yield: 88%.

N,N-d₁₀-Diethyl Acrylamide (d₁₀-DEA)

The synthesis of N,N-d₁₀-diethylacrylamide was performed in the same way as for N,N-d₆-dimethylacrylamide described above, but with the following modifications: N,N-d₁₀-diethylamine (0.5 g, 6 mmol) and triethylamine (1.21 g, 12 mmol), acryloylchloride (0.54 g, 6 mmol) were used instead. The pure product (orange liquid) was analyzed by TLC and NMR. Yield: 56%.

Quantitative Analysis of Single Model Proteins

Bovine serum albumin (BSA) and β-lactoglobulin were used as model proteins. The cysteins in the two proteins were tagged with synthesized and commercial available tagging reagents (see below).

Tagging Reagents:

N,N-Dimethylacrylamide (DMA), Aldrich *
N,N-d₆-Dimethylacrylamide (d6-DMA) *
N,N-Diethylacrylamide (DEA), Dajac *
N,N-d₁₀-Diethylacrylamide (d10-DEA) *
N-Methylacrylamide, Dajac
N-Ethylacrylamide, Dajac
N-Propylacrylamide, Dajac.

BSA (150 μM) and β-lactoglobulin (150 μM) were independently reduced in 1 ml reducing buffer (8M Urea, 50 mM Dithiothreitol (DTT), 50 mM Tris-HCl pH 8.0) for 2 h at 37° C. Each tagging reagent was independently dissolved in 1 ml reducing buffer (described above) to a final concentration of 150 mM. Protein solution (100 μl) and tagging solution (100 μl) were mixed and incubated for 1 hour at 37° C. The concentration of tagging reagent to protein was 1000:1.

The samples were desalted using NAP10 columns (Amersham Pharmacia Biotech, Uppsala, Sweden). The samples were eluted with 1 ml of 50 mM ammonium bicarbonate dissolved in deionized water.

The samples were digested with trypsin (Promega), enzyme:protein mass ratio of ˜1:140, over night in room temperature. BSA and β-lactoglobulin (1 pmol/μl 50% acetonitrile (ACN), 0.5% trifluoroacetic acid (TFA)), tagged with different tags were mixed in varying proportions (table 1). Five combinations of tagging reagents were used for tagging of BSA; (1) DMA (tagging reagent 1), d₆-DMA (tagging reagent 2); (II) DEA (tagging reagent 1), d₁₀-DEA (tagging reagent 2); (III) N-methylacrylamide (tagging reagent 1), N-ethylacrylamide (tagging reagent 2), (IV) N-methylacrylamide (tagging reagent 1), N-propylacrylamide (tagging reagent 2) and (V) N-ethylacrylamide (tagging reagent 1), N-propylacrylamide (tagging reagent 2). β-lactoglobulin was tagged with combination I and II of the tagging reagents.

TABLE 1
Different Volumes of BSA and β-lactoglubulin tagged with
tagging reagent 1 and tagging reagent 2 were mixed to obtain
samples with different concentration ratios of light and
heavy-tagged proteins.

| Tagging reagent 1 | Tagging reagent 2 |
|---|---|
| (μl) | (μl) |
| 80 | 20 |
| 70 | 30 |
| 60 | 40 |
| 50 | 50 |
| 20 | 80 |

After mixing, the samples were dried in a Speed Vac, redissolved in 4 μl matrix solution (α-cyano-4-hydroxycinnamic acid in 50% ACN/0.5% TFA and Angiotensin III: 897.53 m/z and hACTH(18-39): 2465.19 m/z as internal calibrants) and spotted on MALDI-TOF targets.

The samples were analysed by MALDI-TOF and five spectra with 100 shots each were collected per spot/sample. Expected peptides with the actual mass difference were localised in each spectrum and the areas of the peaks were calculated.

Quantitative Analysis of a Mix of Model Proteins

Two vials of Isoelectric Focusing Calibration Protein Mix (Amersham Pharmacia Biotech), each containing: Phosphorylase b (67 μg/vial), Albumin (83 μg/vial), Ovalbumin (147 μg/vial), Carbonic anhydrase (83 μg/vial), Trypsin inhibitor (80 μg/vial), α-Lactalbumin (116 μg/vial) were used for the quantitative analysis. Disulfide bonds of the denatured proteins were reduced by adding 150 μl reducing buffer (described above) to each vial and incubating for 2 h at 37° C. The cysteinyl groups of the proteins in vial one and two were independently tagged with a thousand fold molar excess of DMA and d₆-DMA respectively for 1 h at 37° C. The DMA- and d₆-DMA-tagged proteins were combined as follows (shown as μl of DMA-tagged proteins: μl of d₆-DMA tagged proteins) 25:25, 35:15, 40:10.

The samples were separated by 2D-electrophoresis according the instructions of the manufacturer. The complete samples (190 μg proteins/sample) were applied by cup loading on Immobiline DryStrips (13 cm, pH 3-10, NL (non-linear)) and a MultiPhor II (Amersham Pharmacia Biotech) was used for isoelectric focusing. Second dimension electrophoresis was performed using 12% SDS-PAGE gels.

After separation by 2D-electrophoresis, the gels were fixed in 40% ethanol (EtOH), 10% acetic acid (HAc) for 1 h, stained with, 0.1% Commassie brilliant blue in 40% EtOH, 10% HAc, for 30 min and destained in 20% EtOH, 5% HAc overnight.

One or two of the most intense isoforms of each protein were cut out from the gel using a straw. The gel plugs were washed in 100 μl 50% methanol/50 mM ammonium bicarbonate, repeated three times, followed by evaporation in a Speed Vac. The proteins were digested with trypsin (0.02 μg/50 mM 50% ACN, 0.5% TFA) in room temperature, overnight The peptides were extracted from the gel plugs by adding 100 μl 50% ACN, 0.5% TFA, and incubating 1 h in room temperature repeated two times. The pooled peptide extracts were dried in a Speed Vac and redissolved in 4 μl 50% ACN, 0.5% TFA, respectively. One μl of matrix solution (described above) was mixed with the same volume of sample solution and 0.5 μl of the mixture was deposited on a MALDI target and analysed by MALDI-TOF.

Quantitative Analysis of Proteins of E. coli

Preparation of low speed supernatant of Escherichia coli—Escherichia coli (E. coli), 40 μg (stain B, ATCC 11303, Sigma) was put in 20 ml buffer containing 8M urea, 4% CHAPS, 2% 3-10 pharmalyt (Amersham Pharmacia Biotech) and 65 mM DTT. The cells were disrupted by sonication (Sultrasonics W 385) for 20 seconds repeated 7 times with cooling on ice in between. The lysate was centrifuged 6000×g for 40 min at 8° C. The low speed supernatant (LSS) was stored in −20° C. until used for relative quantification.

Relative quantification—LSS of E. coli was thawed and diluted in reducing buffer (described above) to a final concentration of 5 μg/μl. Two vials, each containing 300 μl diluted LSS were incubated for 2 h at 37° to reduce the disulphide bonds. A volume of 150 μl DMA (38 μg/μl reducing buffer) and d6DMA (38 μg/μl reducing buffer) was added to vial 1 and vial 2 respectively. The vials were incubated for 1 h at 37° to tag the cysteins of the proteins. The proteins of vial 1 and vial 2 were mixed in different ratios (shown as μl of DMA-tagged proteins: μl of di-DMA tagged proteins) 100:100, 140:60, 170:30.

IPG rehydration buffer (8M urea/2% CHAPS/2% IPG buffer 4-7/10 mM DTT) was added to the three samples of different concentration ratios to a final volume of 260 μl. Rehydration of the IPG strips (13 cm, pH 4-7 NL) and 2D-electophoresis were performed following the instructions of the manufacture. The gels were stained with Commassie brilliant blue as described above. Ten protein spots were picked from each gel and destained, digested, extracted and analyzed by MALDI-TOF as described above.

Results

Relative Quantification of BSA and β-lactoglobulin

All tagging reagents described above gave efficient tagging of the cysteins of BSA and β-lactoglobulin. For all combinations of tagging reagents, one or two pairs of mass tagged peptides were identified with baseline separation in the mass spectra. The relative areas within the pairs of the identified peaks were compared with the ratio of the known concentration of light- and heavy-tagged peptides.

Table 1 shows the observed and expected ratios for BSA tagged with DMA/d d₆-DMA and DEA/d₁₀-DEA.

TABLE 1
BSA tagged with DMA, d₆-DMA and DEA, d₁₀-DEA.

| | | |
|---|---|---|
| | Observed ratio | Observed ratio |
| Expected | (DMA/d₆-DMA) | (DEA/d₁₀-DEA) |

| ratio | peak 1 | peak 2 | peak 1 | peak 2 |
|---|---|---|---|---|
| 0.25 | 0.81 | 0.53 | 0.49 | 0.42 |
| 1.0 | 1.21 | 1.14 | 1.13 | 1.07 |
| 1.5 | 1.64 | 1.18 | 1.53 | 2.03 |
| 2.3 | 2.70 | 2.27 | 2.43 | 2.16 |
| 4.0 | 5.04 | 4.30 | 3.28 | 4.08 |

Table 2 shows the observed and expected ratios for β-lactoglobulin tagged with DMA/d₆-DMA and DEA/d₁₀-DEA.

TABLE 2
β-lactoglobulin tagged with DMA, d₆-DMA and DEA, d₁₀-DEA.
(No areas could be calculated for the peaks marked with a hyphen,
due to too weak signals in the mass spectra).

| | | |
|---|---|---|
| | Observed ratio | Observed ratio |
| Expected | (DMA/d₆-DMA) | (DEA/d₁₀-DEA) |

| ratio | peak 1 | peak 2 | peak 1 | peak 2 |
|---|---|---|---|---|
| 0.25 | 0.3 | 0.3 | 0.5 | — |
| 1.0 | 1.0 | 1.0 | 1.1 | — |
| 1.5 | 1.6 | 1.5 | 1.8 | — |
| 2.3 | — | — | — | — |
| 4.0 | 3.5 | 3 | 3 | — |

Table 3 shows the observed and expected ratios for BSA tagged with methylacrylamide, ethylacrylamide and propylacrylamide in the different combinations described above.

TABLE 3
BSA tagged with combinations of methylacrylamide (MA),
ethylacrylamide (EA) and propylacrylamide (PA).

| | Observed Ratio | | Observed Ratio | | Observed Ratio | |
|---|---|---|---|---|---|---|
| Expected | (EA/MA) | | (MA/PA) | | (PA/EA) | |
| Ratio | Peak 1 | peak 2 | peak 1 | peak 2 | peak 1 | peak 2 |
| 1.0 | 1.11 | 1.17 | 0.87 | 1.0 | 0.73 | 0.60 |
| 1.5 | 2.0 | 1.58 | 1.26 | 1.35 | 1.62 | 0.98 |
| 2.3 | 3.58 | 2.26 | 2.02 | 1.43 | 1.49 | 1.14 |
| 4.0 | 3.89 | 5.08 | 2.01 | 1.68 | 2.31 | 2.53 |

Relative Quantification of a Mix of Model Proteins

A mix of six proteins were tagged with DMA and d₆-DMA, separated by 2D-electrophoresis and analysed by MALDI-TOF. Two to three pairs if peptides-tagged with DMA/d₆DMA were detected in the mass spectra of bovine serum albumin, ovalbumin, trypsin inhibitor and α-lactalbumin, respectively.

Table 4 shows the ratios of areas and concentrations for the six proteins in the mix.

TABLE 4
Concentration ratios and area ratios for six proteins tagged with DMA
and d₆-DMA, separated on 2D-eledtrophoresis and analysed by
MALDI-TOF. (No areas could be calculated for the peaks marked
with a hyphen, due to weak signals in the mass spectra).

| | | Ex- | | Observed | Observed |
|---|---|---|---|---|---|
| | | pected | )a Observed | Ratio | ratio |
| | No | ratio | ratio (d0/d6) | (d0/d6) | (d0/d6) |
| Protein | Cys | (d0/d6) | Peak 1 | Peak II | peak III |
| Phosphorylase b | 5 | 1 | — | — | — |
| | | 2.3 | — | — | — |
| | | 4 | — | — | — |
| BSA | 32 | 1 | 1.0:1.0 | 1.0:0.9 | — |
| | | 2.3 | 2.0:1.9 | 2.0:1.9 | — |
| | | 4 | 3.0:4.0 | 3.2:2.8 | — |
| Ovalbumin | 5 | 1 | 1.0:1.0 | 1.1:1.2 | — |
| | | 2.3 | 2.2:2.4 | — | — |
| | | 4 | 3.1:2.9 | 2.9:2.9 | — |
| Carbonic | 0 | | | | |
| anhydrase | | | | | |
| Trypsin | 3 | 1 | 1.0:1.0 | 1.0:1.0 | — |
| inhibitor | | 2.3 | 2.1:2.2 | 2.2:2.2 | |
| | | 4 | 3.6:3.5 | 3.8:3.6 | |
| α-lactalbumin | 7 | 1.1 | 1.0:0.9 | 1.1:1.1 | 1.0:1.0 |
| | | 2.3 | 2.1:2.4 | 2.1:2.1 | — |
| | | 4 | 3.7:3.2 | 3.3:3.6 | — |

Relative Quantification of Proteins of E. coli

Proteins of E. coli tagged with DMA and d6-DMA were combined in three different concentration ratios. The samples were independently separated on 2D-electrophoresis. The same 10 spots were picked from each gel and analysed by MALDI-TOF after digestion with trypsin. The proteins were identified using the ProFound software and theoretical peptide maps were achieved from Swissprot.

The ratios of the areas of the tagged peptides of each protein were calculated and compared with the relative concentrations, table 5. Five of the nine proteins investigated contained cysteins (the total number of 9 proteins is due to that two spots contained the same protein). No untagged cysteins were detected, indicating efficient tagging.

TABLE 5
The second left column shows the theoretical m/z of tagged peptides
of the 10 proteins investigated. The two columns to the right show the
expected and observed ratio of each peptide. (No areas could be
calculated for the peaks marked with a hyphen, due to absence of
or weak signals in the mass spectra).

| | Theoretical m/z | | |
|---|---|---|---|
| Protein | of tagged | | |
| (Accession | peptides | Expected ratio | Observed Ratio |
| number) | (DMA/d₆DMA) | (DMA/d₆DMA) | (DMA/d6DMA) |
| D90738 | — | — | — |
| P11665 | 2085/2091 | 1.0 | 1.0 |
| | | 4.0 | — |
| | | 5.7 | 5.2 |
| | 2044/2050 | 1.0 | 1.0 |
| | | 4.0 | 3.7 |
| | | 5.7 | 5.1 |
| | 1626/1700 | 1.0 | 1.0 |
| | | 4.0 | 2.4 |
| | | 5.7 | 3.9 |
| P11604 | 4966/4972 | 1.0 | — |
| | | 4.0 | — |
| | | 5.7 | — |
| | 4166/4172 | 1.0 | — |
| | | 4.0 | — |
| | | 5.7 | — |
| | 3566/3572 | 1.0 | — |
| | | 4.0 | — |
| | | 5.7 | — |
| | 2760/2766 | 1.0 | 0.9 |
| | | 4.0 | 6.1 |
| | | 5.7 | — |
| P06994 | 2441/2447 | 1.0 | 1.0 |
| | | 4.0 | 3.9 |
| | | 5.7 | 5.4 |
| | 2310/2316 | 1.0 | — |
| | | 4.0 | — |
| | | 5.7 | — |
| P02997 | 2311/2317 | 1.0 | 1.2 |
| | | 4.0 | — |
| | | 5.7 | — |
| | 1012/1018 | 1.0 | — |
| | | 4.0 | — |
| | | 5.7 | — |
| P02927 | — | — | — |
| P07459 | 3735/3741 | 1.0 | — |
| | | 4.0 | — |
| | | 5.7 | — |
| | 2630/2636 | 1.0 | 1.2 |
| | | 4.0 | 2.9 |
| | | 5.7 | 5.9 |
| | 2166/2172 | 1.0 | 1.2 |
| | | 4.0 | 2.5 |
| | | 5.7 | 5.3 |
| | 1814/1826 | 1.0 | 1.0 |
| | | 4.0 | 2.1 |
| | | 5.7 | 4.9 |
| P27291 | — | — | — |
| P32691 | — | — | — |

1. A method for the quantitative determination of the amount of one or more biomolecules in two or more samples by utilising sample unique tagging reagents, characterised in that the method comprises steps (a)-(f)
(a) providing at least two samples;
(b) reacting biomolecules present in each sample with sample unique mass tagging reagents to provide sample unique mass tagged forms thereof;
(c) combining tagged forms present in each sample to provide a single sample;
(d) coseparating, from the resulting sample, a mix of mass tagged forms of each of said biomolecules into different fractions by a separation protocol which is based on a principle selected from the group that consists of differences in hydrophobicity, differences in charge;
differences in isoelectric point (pI); and differences in molecular size;
(e) subjecting, for each fraction, the mix to mass spectrometry to obtain a mass spectrum;
(f) determining from signals in each mass spectrum obtained in step (e) the amount of the biomolecule corresponding to the spectrum in at least one of said samples relative to the amount of the same biomolecule in at least one of the remaining samples.

2. A method according to claim 1, wherein the biomolecule(s) are protein(s) or polypeptide(s) and the reagent is capable of binding to primary amines, cysteine residues or tyrosine residues thereof.

3. A method according to claim 1, wherein the biomolecule(s) are nucleic acid and the reagent capable of binding to cytosine or adenine residues thereof.

4. A method according to any one of claims 1-3, which also includes a digestion step inserted between any one of steps (a)-(e), with the proviso that if the digestion step is between steps (c) and (d), then at least two separate separation protocols are used in step (d).

5. A method according to claim 4, wherein the digestion step is located before step (c).

6. A method according to claim 4, wherein the digestion step is located between steps (d)-(e), preferably with two or more separate separation protocols in step (d).

7. A method according to claim 4, wherein the digestion step is located between steps (a)-(d), such as between steps (a)-(b), (b)-c) or (c)-(d), preferably with two or more separate separation protocols in step (d).

8. A method according to any of claims 1-7, wherein the number (n) of biomolecule(s) to be quantified is two or more.

9. A method according to any of claims 1-8, wherein the number (m) of samples provided in step (a) is three or more.

10. A method according to any of claims 1-9, wherein the biomolecule(s) are digested enzymatically.

11. A method according to any of claims 1-10, wherein two or more separation protocols are used in step (d), and that each of at least two of them are different and capable of separating the mixture of tagged forms derived from step (c) into fractions, in which the predominating one of the biomolecule(s) to be quantified (in tagged forms) or a digestion product thereof (in tagged forms) differs between the fractions.

12. A method according to any of claims 1-11, wherein said sample unique mass tags differ with respect to composition of elements.

13. A method according to any of claims 1-11, wherein said sample unique mass tags differ with respect to isotope composition for at least one element, preferably with the composition of elements being the same.

14. A method according to any of claims 1-13, wherein one of said samples is a reference or a control sample.

15. A method according to any of the claims 1-14, wherein step (d) includes a separation protocol in which the mass transport of the biomolecule(s) to be quantified or of the digestion products thereof is by an applied electric field, such as in electrophoresis, preferably 2D-electrophoresis.

16. A method according to any of the claims 1-14, wherein step (d) includes a separation protocol in which the mass transport of the biomolecule(s) to be quantified or of digestion products thereof is by a liquid flow, such as in chromatography.
